(19) United States
(12) Patent Application Publication
(10) Pub. No.: US 2008/0287359 A1
(43) Pub. Date: Nov. 20, 2008

(54) CADHERIN-11-MEDIATED MODULATION OF HAIR GROWTH

(76) Inventor: Angela M. Christiano, Saddle River, NJ (US)

Correspondence Address: BAKER BOTTS L.L.P., 30 ROCKEFELLER PLAZA, 44TH FLOOR, NEW YORK, NY, 10112-4498, US

(21) Appl. No.: 11/942,333

(22) Filed: Nov. 19, 2007

Related U.S. Application Data
(63) Continuation of application No. PCT/US2006/020646, filed on May 26, 2006.
(60) Provisional application No. 60/685,520, filed on May 27, 2005.
(60) Provisional application No. 60/685,756, filed on May 27, 2005.

(51) Int. Cl. A61K 38/17; A61K 31/7088; A61Q 7/00; A61K 31/7105

(52) U.S. Cl. 514/12; 514/43; 514/44

(57) ABSTRACT

The present invention provides for methods of inhibiting hair growth, comprising decreasing the level of CAD1 1 mRNA and/or protein in hair follicle cells of a subject. The present invention further provides for methods of promoting hair growth, comprising increasing the level of CADI 1 mKNA and/or protein in hair follicle cells of a subject. The invention also provides for transgenic animals with aberrancies in CADI 1 expression, and for assay systems (including transgenic animals and cell-based systems) that may be used to identify agents that decrease or increase CADI 1 expression.

### PRIORITY CLAIM

This application claims priority to U.S. Provisional Applications No. 60/685,520 filed May 27, 2005 and No. 60/685,756 filed May 27, 2005, the contents of each of which are incorporated in their entireties herein.

### BACKGROUND OF THE INVENTION

At the base of each hair, below the surface of the skin, lies the hair follicle. Each hair follicle is comprised of cells from distinct embryonic lineages. During the formation of a hair follicle, mesenchymal cells lying below the surface epithelium induce a small cluster of epithelial cells to form a placode, which in turn directs underlying mesenchymal cells to form the base of the hair follicle, called the dermal papilla. Hair follicles pass through a three stage cycle, consisting of (i) anagen, a growth phase; (ii) catagen, a transitional phase, and (iii) telogen, a resting phase. The dermal papilla, active in growth phase, separates from the rest of the follicle during catagen, and remains in a quiescent state during telogen. The dermal papillae then rejoins the rest of the follicle, and anagen—hair growth—begins again.

The cadherins are a superfamily of transmembrane glycoproteins that mediate cell to cell adhesion in a variety of tissues (Yagi et al., 2000, Genes Dev., 14:1169-1180). A typical cadherin comprises of five extracellular domains, a unique repeated domain also called the cadherin motif or EC domain, containing the negatively charged “Asp-X-Asp”, “Asp-Arg-Glu” and “Asp-X-Asn-Asp-Asn-Ala-Pro-X-Phe” (SEQ. ID. No.: 3) sequence motifs which are involved in calcium binding. The cytoplasmic domain is generally also comprised of highly conserved amino acid residues between different cadherins. The cadherins are responsible for formation of the adherens junction that anchor cells within tissues and organs and to components of the extracellular matrix. Cadherins also contribute to the structure of desmosomes; another stub-cellular structure involved in cell to cell anchoring. Upon calcium ion binding by the extracellular region, the extracellular domains of cadherins change conformation, allowing lateral dimerization and subsequent cross-association between dimers on adjacent cells. Usually the interactions are between similar subspecies of cadherins. The cytoplasmic tail of cadherins interact directly or indirectly with “linker proteins,” including β-catenin, plakoglobin or p120,-catenin, -actinin, and vinculin. These proteins are thought to couple cadherins to the actin cytoskeleton to strengthen the adhesive force of the entire junctional complex (Steinberg e al., 1999, Curr. Opin. Cell Biol. 11:554-560).

There are at least 80 members of the cadherin superfamily and while most are transmembrane proteins, exceptions do exist. Of the “classic-cadherins” comprising Cadherins E (epithelial), N (neuronal), P (placental), R, VE, K, T1, T2, 11 or OB (osteoblast), H, M (muscle), KSP, and LI (Yagi et al., 2000, Genes Dev., 14:1169-1180), E-cadherin is best-characterized at the molecular as well as biologically functional level (Yagi et al., 2000, supra). Differential expression of E-cadherin has been implicated in several aspects of development, including cell sorting during gastrulation and tissue morphogenesis as well as the establishment of differentiated cell identity. While decreased E-cadherin expression has been correlated with metastasis and decreased survival in several different cancers, including breast carcinoma, its loss is not an absolute predictor of tumor invasion or metastasis. Increased expression of other cadherins, such as N-cadherin and P-cadherin, may also be associated with development or progression of breast carcinoma.

Cadherin-11, or OB (osteoblast)-cadherin, was originally identified in mouse osteoblasts (Okazaki et al., 1994, J. Biol. Chem., 269(16):2092-12098) but was later found to be expressed in a variety of normal tissues of mesodermal origin, including areas of the kidney and brain (Hoffmann et al., 1995, Dev. Biol., 169:337-346). Generally, the identity of amino acid sequences is highest in the putative cytoplasmic domain followed by EC1 and EC2 domains, when comparison of known cadherin molecules is made. This general trend of clustered, more prominent similarity in the regions mentioned above is observed when cadherin 11 amino acid sequence is compared with other cadherin molecules. Cadherin 11 contains almost all amino acid sequence motifs shared in the cadherin family including the motifs containing negatively charged amino acids such as DXD, DXE, or DXNDN (SEQ. ID. No.: 4) in the extracellular domain, are well conserved in the internal repeats. In addition, the four cysteine residues that are present in the EC5 domain of known cadherins are all conserved in cadherin 11. Similarly, a 70 amino acid residue cytoplasmic domain at the C-terminus is strongly conserved indicating that cadherin 11 has catenin binding sites. Although classified as a type II cadherin (lacking a conserved HAV sequence), cadherin-11 is otherwise similar in structure to the type I cadherins N- and P-cadherin. In addition, the genomic structure of cadherin-11 exhibits a unique mRNA splice site, allowing for two forms of the protein to be expressed. Cadherin-11 mRNA is expressed in several types of cancer cells, including colon, gastric, renal cell, and breast cancer and osteosarcoma. In many instances, cadherin-11 expression has been associated with more aggressive, dedifferentiated cancers, such as the signet ring cell subtype of gastric carcinoma (Shimazui et al., 1996 Cancer Res. 56:3234-3237).

Simonneau et al. (1995, Cell Adhes. Commun. 3(2)115-130) reported that, in the developing rat embryo, cadherin-11 (“CAD11”) expression was found to be specific to the mesenchymal phenotype, and was expressed in epithelium to mesenchyme inductions that operate in the nasal septum, skin, vibrissae, teeth, and various glands.

Nanba et al. (2003, Anat. Record A 270A:97-102) reported that intercellular junctions containing cadherin-11 (“CAD11”) are established in the dermal papillae of the developing hair follicle. Although CAD11 was, at the time, the only member of the CAD family reported to be expressed in dermal papillae, because it had been reported that mice lacking CAD11 (−/−) have normal hair growth (Horikawa et al., 1999, Dev. Biol. 215:182-189; Manabe et al., 2000, Mol. Cell. Neurosci. 15:534-546), Nanba suggests that multiple cadherins are likely to be involved in dermal papilla aggregation.

### SUMMARY OF THE INVENTION

The present invention provides for methods and compositions for inhibiting hair growth by decreasing the level and/or activity of CAD11 mRNA and/or protein. The present invention further provides for methods and compositions for promoting hair growth by increasing the level and/or activity of CAD11 mRNA and/or protein. This invention is based, at least in part, on the discovery that levels of CAD11 protein decreased over time in cultures of human dermal papillae, where the decrease correlates with a loss in the ability of the dermal papillae to induce hair follicle formation.

In a first set of embodiments, the present invention provides for methods of promoting hair growth comprising increasing the level and/or activity of CAD11 mRNA and/or protein in cells, preferably hair follicle cells, and more preferably dermal papilla cells, of a subject. The level of CAD11 may be increased either directly, for example by introducing, into a hair follicle cell, CAD11 mRNA or protein, or it may be increased indirectly, by providing an agent that results in increased expression of an endogenous CAD11 gene or increased functional activity of CAD11 protein.

In a second set of embodiments, the present invention provides for methods for identifying agents that may be used to inhibit hair growth, comprising exposing an appropriate test cell or organism to a test agent, and then determining whether expression of CAD11 is decreased relative to the level of CAD11 in a control cell or organism not exposed to the test agent. In an alternate set of embodiments, the present invention provides for methods for identifying agents that may be used to promote hair growth, comprising exposing an appropriate test cell or organism to a test agent, and then determining whether expression of CAD11 is increased relative to the level of CAD11 in a control cell or organism not exposed to the test agent. The ability of an agent to decrease CAD11 indicates that it may be used to inhibit hair growth. Likewise, the ability of the agent to increase the level of CAD11 indicates that the agent may be used to promote hair growth.

In a third set of embodiments, the present invention provides for a transgenic non-human animal containing a transgene which interrupts or otherwise disrupts expression of at least one CAD11 gene, including (i) so-called “knock-out” animals as well as (ii) animals in which the transgene encodes an antisense CAD11 nucleic acid operably linked to a promoter element, wherein the promoter element may be constitutively active or inducible in hair follicle cells of the animal. Such transgenic animals may be used to study the relationship between CAD11 expression and hair growth, and may be used in screening methods to identify agents that modulate CAD11 expression.

In a fourth set of embodiments, the present invention provides for a transgenic non-human animal containing a transgene comprising a CAD11 gene, operably linked to a promoter element, wherein the promoter element may be constitutively active or inducible in hair follicle cells of the animal. Such transgenic animals may be used to study the relationship between CAD11 expression and hair growth, and may be used in screening methods to identify agents that increase CAD11 expression.

In a fifth set of embodiments, the present invention provides for compositions that may be used to inhibit hair growth, which may comprise CAD11-directed antisense, siRNA, and/or catalytic nucleic acids and/or agents that indirectly inhibit CAD11 expression.

In a sixth set of embodiments, the present invention provides for compositions that may be used to promote hair growth, which may comprise CAD11 nucleic acid and/or protein, agents that indirectly increase CAD11 expression, and/or hair follicle cells in which CAD11 expression is increased or which have been administered CAD11 protein.

In a seventh set of embodiments, the present invention provides for a method of treating alopecia in a subject comprising administering to cells, preferably hair follicle cells, of the subject, an agent that increases the level of CAD11 mRNA and/or protein in at least a proportion of cells to which it is administered. In non-limiting specific embodiments, the alopecia being treated is male pattern baldness in a human.

In an eighth set of embodiments, the present invention provides for methods of inhibiting hair growth comprising decreasing levels of CAD11 mRNA and/or protein in cells, preferably hair follicle cells, of a subject. In specific, non-limiting embodiments, levels of CAD11 expression may be decreased by administering, to hair follicle cells, RNAi, antisense RNA, or catalytic nucleic acids that comprise regions that are complementary to CAD11 mRNA.

### BRIEF DESCRIPTION OF THE FIGURES

FIG. 1A-B. Photomicrographs of HaCaT cells transfected with an expression construct encoding a CAD11-GFP fusion protein, showing green fluorescence at cell borders (white). Nuclei are stained with DAPI (dark gray).

FIG. 2A-C. Immunuofluorescent staining of CAD11 in rat anagen vibrissa (light gray); Nuclei are stained with DAPI (dark gray). (2A) 10× magnification; (2B) 20× magnification; (2C) 40× magnification.

FIG. 3. Western blot of protein samples from human dermal papilla cultures stained with antibody to CAD11. Control (3C) is E14.5 mouse whole embryo lysate. P2-P8 are protein samples from human dermal papilla cultures passaged 2-8 times, respectively.

FIG. 4A-D. Immunofluorescent staining of CAD11 in developing dermal papillae of embryonic rat (light gray). Nuclei are stained with DAPI (dark gray). (A) Embryonic day 14.0 (E14.0); (B) Embryonic day 15.5 (E15.5); (C) Embryonic day 16.5 (E16.5); and (D) Embryonic day 17.5 (E17.5).

FIG. 5A-F. Immunofluorescent staining of CAD11 in postnatal rat dermal papillae (light gray). Nuclei are stained with DAPI (dark gray). (A) Postnatal day 1 (P1); (B) Postnatal day 7 (P7); (C) Postnatal day 10 (P10); (D) Postnatal day 22 (P22); (E) Postnatal day 25 (P25); (F) Postnatal day 30 (P30).

FIG. 6A-C. Nucleic acid sequence of human CAD11 cDNA (SEQ ID NO:1), NCBI Acc. No. NM_{—}001797. (A) Nucleic acid residues 1-1740; (B) Nucleic acid residues 1741-3540; (C) Nucleic acid residues 3541-3654.

FIG. 7. Amino acid sequence of human CAD11 protein (SEQ ID NO:2), NCBI GenBank Acc. No. NP_{—}001788, NM_{—}001797. The preprotein is residues 1-796; the signal peptide is residues 1-22; the proprotein is residues 23-796 and the mature protein is residues 54-796.

### DETAILED DESCRIPTION OF THE INVENTION

For clarity of presentation, and not by way of limitation, the detailed description of the invention is divided into the following subsections:
(i) transgenic animals;
(ii) assay systems; and
(iii) methods of decreasing CAD11 in a subject;
(iv) methods of directly increasing CAD11 in a subject; and
(v) methods of indirectly increasing CAD11 in a subject.

The present invention may be applied to a human or a non-human subject. Non-limiting examples of non-human subjects that may benefit from the invention include domesticated farm, companion or laboratory animals (especially mammals such as cats, dogs, rabbits, ferrets, guinea pigs, rats, mice and hamsters) as well as animals that are used in the wool (e.g., sheep, alpaca, llama) or fur industries.

As used herein, the term CAD11, without more, can refer to the CAD11 gene, CAD11 cDNA, CAD11 mRNA, CAD11 protein, or a combination thereof.

The present invention encompasses CAD11 nucleic acids and proteins of diverse species.

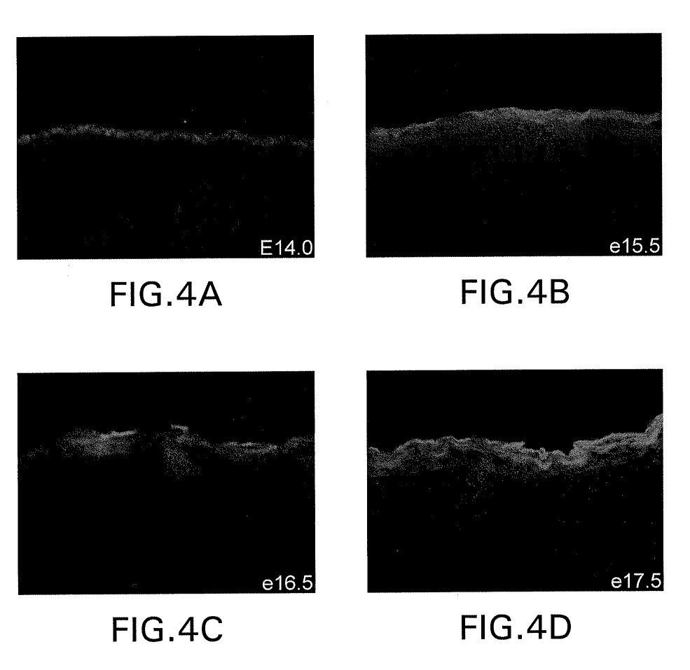

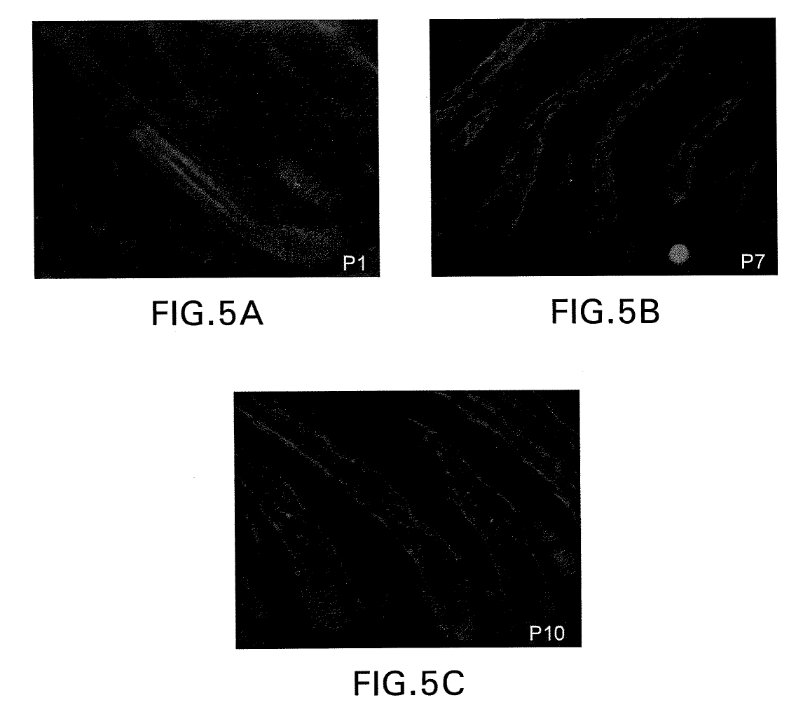

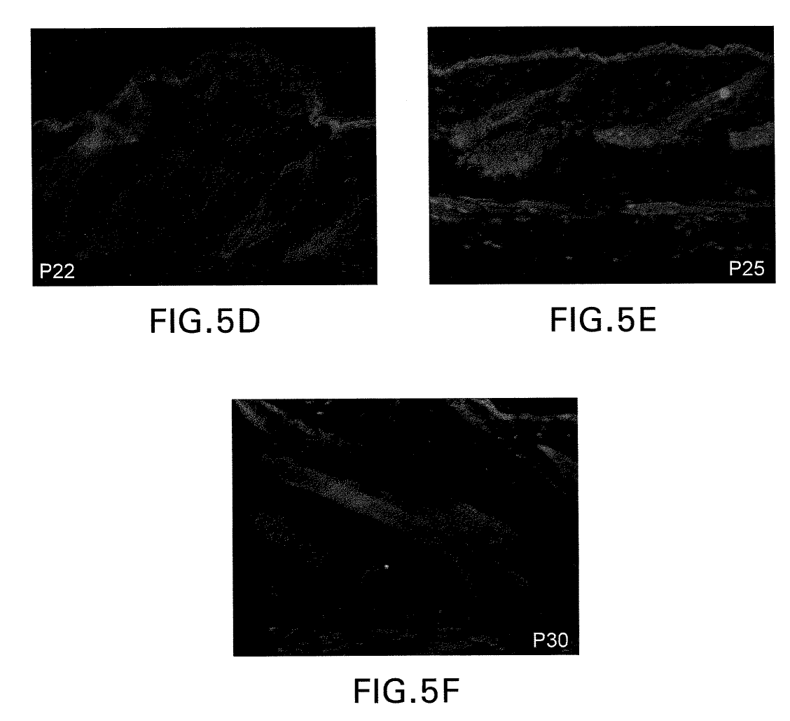

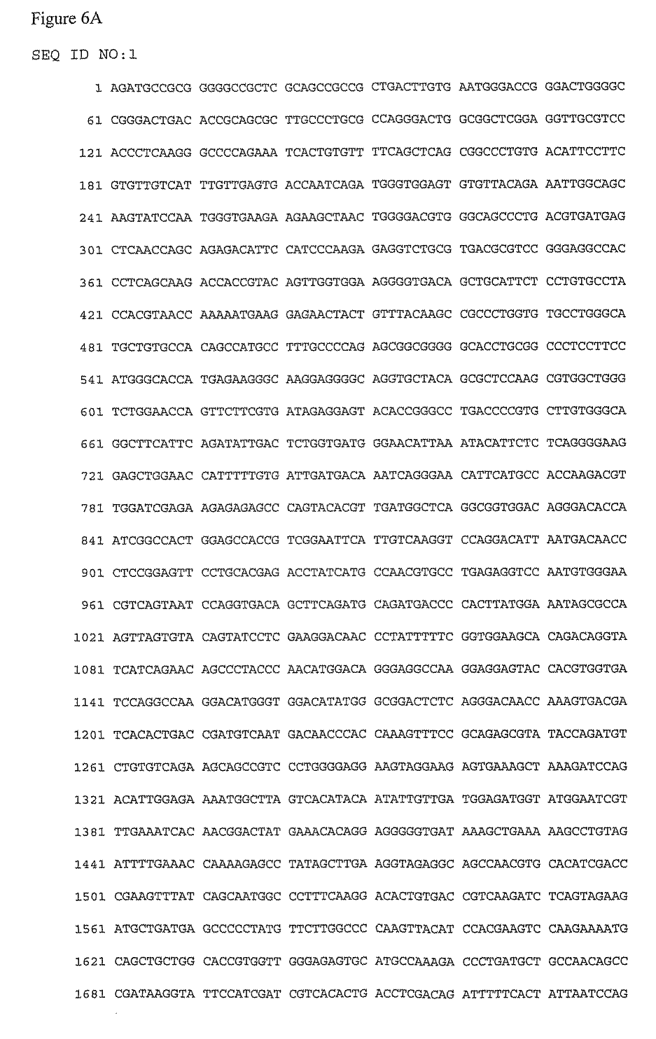

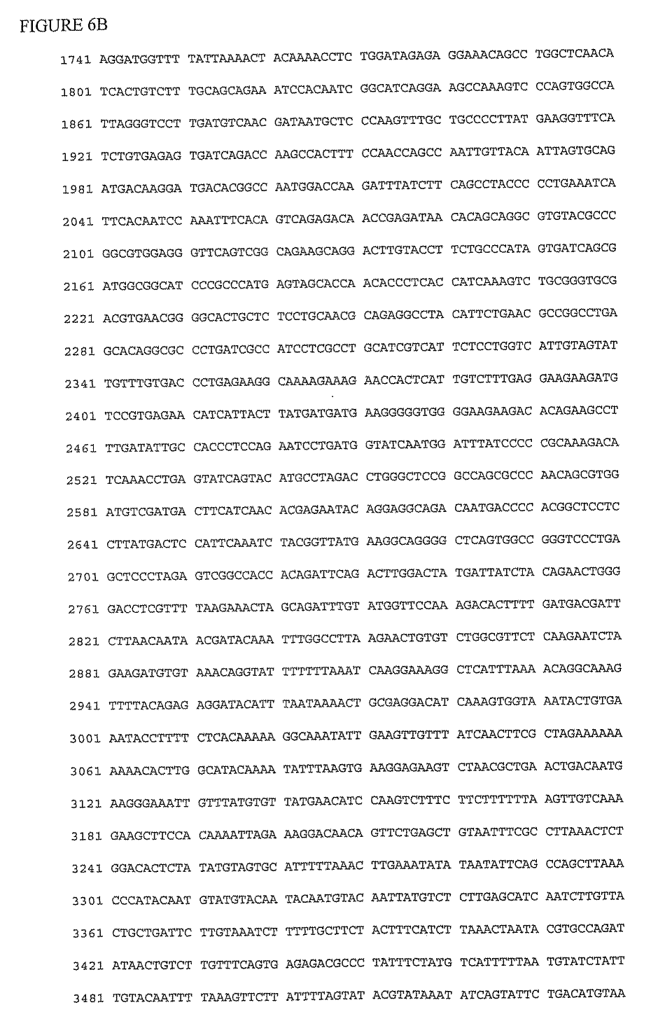

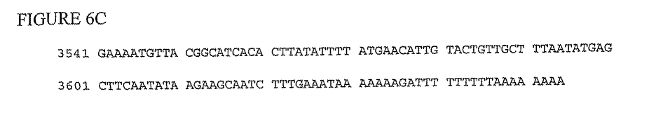

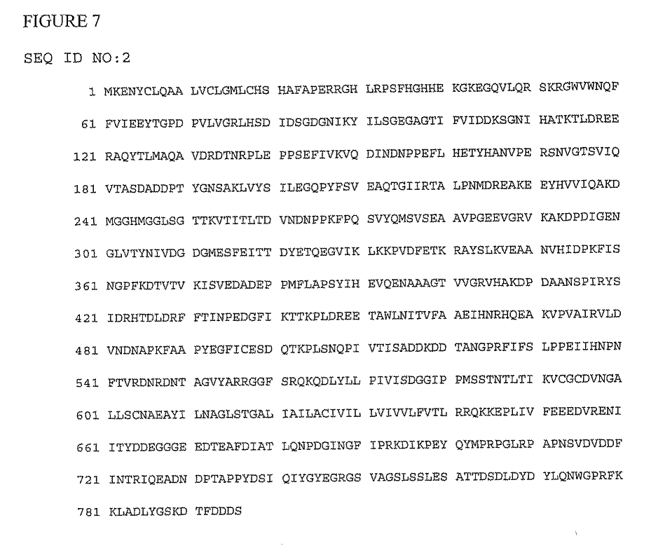

In particular non-limiting embodiments, the present invention relates to a human CAD11 nucleic acid having a sequence set forth in SEQ ID NO:1 and FIG. 4 with GenBank Accession number NM_{—}001797, and in particular to the coding region of the gene, as set forth in SEQ ID NO:5, as well as to nucleic acids at least about 12, 15, 20, 25, 50, 100, 200, 500, 1000, 1500, 2000, 2500, 3000, 3500, 3600, or 3655 nucleotides in length that either (i) are at least about 85, 90, or 95 percent homologous to a comparable length of nucleic acid of SEQ ID NO: 1, SEQ ID NO:5, or their respective complement (as determined using standard software such as BLAST or FASTA) and/or (ii) hybridize to a comparable length of nucleic acid having a sequence as set forth in SEQ ID NO: 1, SEQ ID NO:5, or their respective complement under stringent conditions, defined as e.g., hybridization in 0.5 M NaHPO₄, 7 percent sodium dodecyl sulfate (“SDS”), 1 mM ethylenediamine tetraacetic acid (“EDTA”) at 65° C., and washing in 0.1×SSC/0.1 percent SDS at 68° C. (Ausubel et al., 1989, Current Protocols in Molecular Biology, Vol. I, Green Publishing Associates, Inc., and John Wiley & Sons, Inc. New York, at p. 2.10.3). The present invention further provides for nucleic acids encoding a CAD11 protein having a sequence as set forth in SEQ ID NO:2, FIG. 5. In other non-limiting embodiments, the present invention relates to nonhuman CAD11 nucleic acids such as Mus musculus CAD11 (GenBank accession number NM_{—}009866). Nucleic acids of the invention may be DNA, RNA, or cDNA, and may be single or double stranded, and include, within their scope, siRNA, antisense RNA, ribozyme and deoxyribozyme molecules.

The present invention further provides for CAD11 proteins, defined as proteins encoded by any of the CAD11 nucleic acids of the preceding paragraph. In specific, nonlimiting embodiments, the invention relates to a human CAD11 protein having a sequence as set forth in SEQ ID NO:2 and FIG. 5 with GenBank accession numbers NP_{—}001788 and NM_{—}001797, as well as to the proprotein or mature protein having residues 23-796 or 54-796 of SEQ ID NO:2, respectively, as well as to proteins or peptides at least about 12, 15, 22, 25, 50, 100, 150, 200, 250, 300, 350, 400, 450, 500, 550, 600, 650, 700, 740, 742, 750, 760, 770, 773, 780, 790, or 796 amino acid residues in length that are at least about 85, 90, or 95 percent homologous to a comparable length of sequence of SEQ ID NO:2 (as determined using standard software such as BLAST or FASTA). The present invention also encompasses non-human CAD11 proteins such as Mus musculus CAD11 GenBank accession numbers NP_{—}033996 and NM_{—}009866), and nucleic acids encoding said proteins.

### Transgenic Animals

The present invention provides for transgenic animals that contain a transgene that results in altered expression levels of CAD 11. In one set of non-limiting embodiments, the transgene is a CAD11-encoding nucleic acid in antisense orientation, operably linked to a promoter element which may optionally be selectively or specifically active in hair follicle cells or be inducible. In another set of non-limiting embodiments, the transgenic animal is a CAD11 “knock-out” animal, in which the expression of at least one CAD11gene is inhibited by the introduction of foreign DNA in the CAD11 gene or the chromosomal region containing the CAD11 gene. In yet another set of non-limiting embodiments, the transgene is a CAD11-encoding nucleic acid, operably linked to a promoter element, where the promoter element may optionally be selectively or specifically active in hair follicle cells or be inducible.

Non-limiting examples of promoter elements that are specifically or selectively expressed in hair follicle cells include the versican promoter (Naso et al., 1994, J. Biol. Chem. 269(52):32999-33008; Kishimoto et al., 1999, Proc. Natl. Acad. Sci. U.S.A. 96(13):7336-7341); the fibroblast growth factor 18 promoter (Kawano et al., 2005, J. Invest. Dermatol. 124(5):877-885; Shimokawa et al., 2003, Cancer Res. 63:6116-6120); the osteopontin promoter (Yu et al., 2001, J. Invest. Dermatol. 117(6):1554-1558; Wang et al., 2000, Oncogene 19(50):5801-5809; Tezuka et al., 1996, J. Biol. Chem. 271(37):22713-22717); and the prolactin promoter (Foitzik et al., 2003, Am. J. Pathol. 162(5):1611-1621; Takasuka et al., 1998, Endocrinology 139(3):1361-1368; Maurer et al., 1989, J. Biol. Chem. 264(12):6870-6873).

In specific, non-limiting embodiments, the present invention provides for transgenic animals containing a transgene which is CAD11 linked to one of the foregoing promoters. In a preferred, non-limiting embodiment, the present invention provides for a transgenic mouse containing a transgene which is CAD11, which may be murine or human CAD11, operably linked to a versican promoter.

Trangenic animals which may be produced according to the invention include, but are not limited to, transgenic mice, rats, goats, sheep, cats, and pigs. Standard techniques in the construction and analysis of transgenic animals may be performed as described in manuals such as “Manipulating the mouse embryo, A laboratory manual” 3rd Edition, 2003 (Nagy et al. Eds., pp. 289-358; Cold Spring Harbor Laboratory Press, Cold Spring Harbor, N.Y.). Transgenic animals may also be constructed by microinjection or electroporation of embryonic stem cells. The procedure to construct a transgenic line from an ES cell is also well known in the art and may be performed for example as described in manuals such as Nagy et al., 2003, supra.

In non-limiting embodiments wherein the transgenic animal is a CAD11 “knockout,” targeted disruption of a gene to generate a null or mutated allele is usually accomplished by insertion of a selectable marker (usually neomycin) into a gene causing disruption of splicing, promoter function, or reading frame, with or without deletion of some of the gene. Incorporation of the altered gene into the mouse genome depends upon replacement of the endogenous gene by homologous recombination through both of the arms of the altered gene into one allele of genomic DNA. Methods to prepare a mouse line with a targeted disruption or knockout of a specific gene is well known to the art and may for example be performed by routine techniques described in manuals such as Nagy et al., 2003, supra.

Conditional knockout mice may be prepared using the Cre-loxP System. When global removal of a gene of interest using conventional knockout methods results in embryonic lethality, a conditional knockout line of mice may be produced where tissue-specific deletions can still be studied in vivo. To knock out a gene in a specific tissue, an important exon of the targeted gene is subcloned in between two loxP sites (i.e., clone loxP elements in each of the introns flanking the exon to be deleted). Homologous recombinants are generated by a homologous recombination replacement event as with conventional targetings described above. In such animals the gene will retain normal function in the ES cells and in the parental chimeras and agouti heterozygotes and homozygotes, thus allowing the mice to survive and breed. After germline transmission has occurred and the mice are bred to homozygosity, the “floxed” mice may be bred to a tissue-specific Cre transgenic mouse, and in the resulting offspring, the exon will be removed only in that tissue. Thus the effect of ablation a gene activity may be studied in a cell, tissue or organ specific manner and the problem or embryonal lethality due to loss of the targeted gene is overcome. Conditional knockout mice may be constructed by methods well known to the art and may for example be performed by routine techniques described in manuals such as Nagy et al., 2003, supra.

### Assay Systems

The present invention provides for assay systems that may be used to identify agents that decrease or increase CAD11 expression. The present invention further provides for assay systems that may be used to identify agents that increase CAD11 expression. The assay systems may utilize isolated cells, multicellular structures, or organisms, including, but not limited to, the transgenic animals described above.

In particular non-limiting embodiments, the present invention evaluates the effects of test agents on endogenous CAD11 expression. Accordingly, the present invention provides for a method of identifying an agent that inhibits hair growth, comprising administering, to a test cell or a test organism, a test agent, and determining whether the test agent induces a decrease in the level of CAD11 expression, as measured by mRNA or protein, which may be performed using standard techniques including, but not limited to, PCR, Northern blot, Western blot, etc. In alternate non-limiting embodiments, the present invention evaluates the effects of test agents on endogenous CAD11 expression. Accordingly, the present invention provides for a method of identifying an agent that promotes hair growth, comprising administering, to a test cell or a test organism, a test agent, and determining whether the test agent induces an increase in the level of CAD11 expression, as measured by mRNA or protein, which may be performed using standard techniques including, but not limited to, PCR, Northern blot, Western blot, etc.

The change in CAD11 level may be measured relative to the CAD11 level in a control cell or organism not exposed not test agent. A test agent that decreases CAD11 levels is likely to inhibit hair growth, and may optionally be administered to a test subject to determine whether hair growth is inhibited in the test subject, Similarly, a test agent that increases CAD11 levels is likely to promote hair growth, and may optionally be administered to a test subject to determine whether hair growth is promoted in the test subject.

Transgenic animals in which CAD11 is overexpressed may be used in analogous assay systems to identify agents which inhibit CAD11 expression or activity. Likewise, transgenic animals in which CAD11 is underexpressed, relative to wild-type animals, may be used in analogous systems to identify agents that increase CAD11 expression or activity. “Activity,” as used in this paragraph, refers to the ability of CAD11 to promote hair growth, where an agent is envisioned which may not increase expression of CAD11 but which may render the endogenous CAD11 protein more potent in promoting hair growth.

### Methods of Decreasing CAD11 in a Subject

The present invention further provides for methods of decreasing CAD11 expression in a subject. Such a decrease may be effected, for example, by administering, to a subject in need of such treatment, either an agent identified as decreasing CAD11 mRNA and/or protein, using an assay system as set forth above, or by administering an effective amount of an siRNA, antisense RNA, or catalytic nucleic acid (e.g., ribozyme or deoxyribozyme) directed at CAD11 mRNA.

In non-limiting embodiments of the invention, expression of the CAD11gene may be decreased using a ribozyme, i.e., an RNA molecule with catalytic activity. See, e.g., Cech, 1987, Science 236:1532-1539; Cech, 1990, Annu. Rev. Biochem. 59:543-568; Cech, 1992, Curr. Opin. Struct. Biol. 2:605-609; Couture and Stinchcomb, 1996, Trends Genet. 12:510-515. Ribozymes can be used to inhibit CAD11 gene function by cleaving an RNA sequence, as is known in the art (e.g., Haseloff et al., U.S. Pat. No. 5,641,673). The 5′ untranslated, 3′ untranslated or coding sequence of the CAD11 gene may be used to generate a ribozyme which will specifically bind to mRNA transcribed from the CAD11 gene. Methods of designing and constructing ribozymes which can cleave other RNA molecules in trans in a highly sequence specific manner have been developed and described in the art (see, Haseloff et al., 1988, Nature 334:585-591). In a non-limiting embodiment, the nucleic acid length for a catalytic nucleic acid is between about 10 and 500, or between about 13 and 200, or between about 13 and 100 nucleotides.

In other non-limiting embodiments of the invention, expression of the CAD11 gene may be inhibited using an antisense oligonucleotide sequence. The antisense sequence is complementary to at least a portion of the 5′ untranslated, 3′ untranslated or coding sequence of the CAD11 gene. Preferably, the antisense oligonucleotide sequence is at least six nucleotides in length, but can be up to about 50 nucleotides long. Longer sequences can also be used. The antisense oligonucleotides of the invention may be DNA, RNA, or any modifications or combinations thereof. As an example of the modifications that the oligonucleotides may contain, inter-nucleotide linkages other than phosphodiester bonds, such as phosphorothioate, methylphosphonate, methylphosphodiester, phosphorodithioate, phosphoramidate, phosphotriester, or phosphate ester linkages (Uhlman et al., 1990, Chem. Rev. 90(4):544-584; Tidd, 1990, Anticancer Res. 10(5A):1169-1182), may be present in the oligonucleotides, resulting in their increased stability. Oligonucleotide stability may also be increased by incorporating 3′-deoxythymidine or 2′-substituted nucleotides (substituted with, e.g., alkyl groups) into the oligonucleotides during synthesis, by providing the oligonucleotides as phenylisourea derivatives, or by having other molecules, such as aminoacridine or poly-lysine, linked to the 3′ ends of the oligonucleotides (see, e.g., Tidd, 1990, supra). Modifications of the RNA and/or DNA nucleotides comprising the oligonucleotides of the invention may be present throughout the oligonucleotide, or in selected regions of the oligonucleotide, e.g., the 5′ and/or 3′ ends. The antisense oligonucleotides may also be modified so as to increase their ability to penetrate the target tissue by, e.g., coupling the oligonucleotides to lipophilic compounds. The antisense oligonucleotides of the invention can be made by any method known in the art, including standard chemical synthesis, ligation of constituent oligonucleotides, and transcription of DNA encoding the oligonucleotides, as described below. Precise complementarity is not required for successful duplex formation between an anitisense molecule and the complementary coding sequence of the CAD11 gene. Anitisense molecules which comprise, for example, 2, 3, 4, or 5 or more stretches of contiguous nucleotides which are precisely complementary to a portion of a coding sequence of the CAD11 gene, each separated by a stretch of contiguous nucleotides which are not complementary to adjacent coding sequences, can provide targeting specificity for mRNA of the CAD11 gene. Preferably, each stretch of contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and the CAD11 gene untranslated or coding sequence.

In further non-limiting embodiments of the invention, dsRNA-mediated interference (RNAi or siRNA), which is well known in the fields of molecular biology, may be used to inhibit the expression of the CAD11 gene (see, for example, Hunter, 1999, Curr. Biol. 9:R440-442; Hamilton et at., 1999, Science 286:950-952; Ding, 2000, Curr. Opin. Biotechnol. 11:152-156). siRNA typically comprises a polynucleotide sequence identical or homologous to a target gene (or fragment thereof) linked directly, or indirectly, to a polynucleotide sequence complementary to the sequence of the target gene (or fragment thereof). The dsRNA may comprise a polynucleotide linker sequence of sufficient length to allow for the two polynucleotide sequences to fold over and hybridize to each other; however, a linker sequence is not necessary. The linker sequence is designed to separate the anitisense and sense strands of siRNA sufficiently as to limit the effects of steric hindrance and allow for the formation of dsRNA molecules and should not hybridize with sequences within the hybridizing portions of the dsRNA molecule. Accordingly, one method for inhibiting hair growth comprises the use of (siRNA) comprising polynucleotide sequences identical or homologous to the CAD11 gene. In one non-limiting embodiment, the nucleic acid length for an antisense or siRNA molecule is between about 6 and 50, or between about 10 and 35, or between about 10 and 25, or between about 15 and 25 nucleotides.

RNA containing a nucleotide sequence identical to a fragment of the target gene is preferred for inhibition; however, RNA sequences with insertions, deletions, and point mutations relative to the target sequence can also be used for inhibition. As described above for antisense molecules, sequence identity may optimized by sequence comparison and alignment algorithms known in the art (see, Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

Preferably, siRNA is targeted to a polynucleotide sequence of the CAD11gene. Preferred siRNA molecules of the instant invention are highly homologous or identical to the polynucleotides encoding the CAD11 gene. The homology may be greater than 70%, preferably greater than 80%, more preferably greater than 90% and is most preferably greater than 95%. In one preferred embodiment, the siRNA and antisense molecule is targeted to the coding region of CAD11 as defined in SEQ. ID. No.: 5.

Ribozymes, antisense polynucleotides, and siRNA molecules may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands); the promoters may be known inducible promoters such as baculovirus. Inhibition may be targeted by specific transcription in an organ, tissue, or cell type. The RNA strands may or may not be polyadenylated; the RNA strands may or may not be capable of being translated into a polypeptide by a cell's translational apparatus.

RNA may also be chemically or enzymatically synthesized by manual or automated reactions. The RNA may be synthesized by a cellular RNA polymerase or a bacteriophage RNA polymerase (e.g., T3, T7, SP6). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no, or a minimum of, purification to avoid losses due to sample processing. The RNA may be dried for storage or dissolved in an aqueous solution. The solution may contain buffers or salts to promote annealing, and/or stabilization of the duplex strands.

Ribozymes, antisense molecules, and siRNA can be introduced into cells as part of a DNA construct, as is known in the art. The DNA construct can also include transcriptional regulatory elements, such as a promoter element, an enhancer or UAS element, and a transcriptional terminator signal, for controlling the transcription of the ribozyme in the cells. Mechanical methods, such as microinjection, liposome-mediated transfection, electroporation, or calcium phosphate precipitation, can be used to introduce such DNA constructs into cells whose division it is desired to decrease, as described above. Alternatively, if it is desired that the DNA construct be stably retained by the cells, the DNA construct can be supplied on a plasmid and maintained as a separate element or integrated into the genome of the cells, as is known in the art.

Administration of one or more agent that decreases CAD11may be achieved by methods known in the art, including topical, intracutaneous, intravenous, or oral administration. In preferred non-limiting embodiments of the invention, administration is targeted to hair follicle cells to be treated. The one or more agent may be comprised in a composition together with suitable pharmaceutical carriers. The agent may optionally be comprised in a microstructure such as a liposome or microsphere. A composition comprising the agent may further comprise a permeability-enhancing agent such as dimethylsulfoxide, lipofectamine, oligofectamine, nanoparticles, and/or cyclofectin.

One or more agent may be administered as a single application or as multiple applications over a period of time. Administration of agent may be, for example and not by way of limitation, once or twice daily, once or twice weekly, once a month, twice a month, once every two months, or once a year. Alternatively, the agent(s) may be administered for a treatment period, followed by a period of no treatment, followed by another treatment period, and this cycle may be repeated. In a specific, non-limiting embodiment of the invention, the interval between treatments or treatment periods may be the average length of time, in the subject to be treated, for a hair follicle to pass through one cycle.

According to the invention, one or more agent that inhibits CAD11 expression may be administered ex vivo to a hair follicle cell, which may be an isolated cell or a cell in a hair follicle and/or dermal papilla in culture. Methods of harvesting hair follicles and dermal papillae, and maintaining them in culture, are known in the art,

The foregoing methods of decreasing levels of CAD11by administering an effective amount of one or more agent may be used to inhibit hair growth in a subject in need of such treatment. In non-limiting embodiments, such methods may be used to treat hypertrichosis, or may be used as cosmetic depilatory agents. In other embodiments, such methods may be used to inhibit hair growth in a domesticated animal which is a laboratory animal, farm animal, pet, or which is used in the leather industry.

### Methods of Directly Increasing CAD11 in a Subject

The present invention provides for methods of directly increasing CAD11 in a cell of a subject comprising introducing, into said cell, a nucleic acid encoding CAD11 in expressible form or introducing, into said cell, CAD11 protein. Preferably, but not by way of limitation, the cell is a hair follicle cell, where the hair follicle cell may be comprised in the skin of a subject or may be an isolated cell or may be part of an isolated multicellular structure (e.g., an isolated follicle, a dermal papilla or a placode). In specific, non-limiting embodiments, the hair follicle cell is a dermal papilla cell. In a non-limiting embodiment, the nucleic acid encoding CAD11 comprises a CAD11 coding sequence as identified in SEQ. ID. No.: 5.

Where CAD11 expression is increased by introducing a CAD11 encoding nucleic acid into a cell, the nucleic acid may be operably linked to a promoter element such as a heterologous promoter element (i.e., not the endogenous CAD11 promoter), and optionally to other elements that aid in transcription and/or translation. The promoter element may be selectively or specifically active in a hair follicle cell, or may be expressed in diverse tissues. Alternatively, the promoter element may be inducible. Non-limiting examples of promoter elements that are specifically or selectively expressed in hair follicle cells include the versican promoter (Naso el al., 1994, J. Biol. Chem. 269(52):32999-33008; Kishimoto et al., 1999, Proc. Natl. Acad. Sci. U.S.A. 96(13):7336-7341); the fibroblast growth factor 18 promoter (Kawano et al., 2005, J. Invest. Dermatol. 124(5):877-885, Shimokawa et al., 2003, Cancer Res. 63:6116-6120); the osteopontin promoter (Yu et al., 2001, J. Invest. Dermatol. 117(6):1554-1558; Wang et al., 2000, Oncogene 19(50):5801-5809; Tezuka et al., 1996, J. Biol. Chem. 271(37):22713-22717); and the prolactin promoter (Foitzik et al., 2003, Am. J. Pathol. 162(5):1611-1621; Takasuka et al., 1998, Endocrinol. 139(3):136101368; Maurer et al., 1989, J. Biol. Chem. 264(12):6870-6873). Non-limiting examples of promoter elements that are inducible include tetracycline inducible promoters and metallothionine inducible promoters. Other non-limiting examples of promoters that may be used according to the invention include the cytomegalovirus immediate early promoter.

A CAD11 encoding nucleic acid, in expressible form (e.g., operably linked to a promoter element), may optionally be comprised in a vector molecule. Suitable expression vectors include virus-based vectors and non-virus based DNA or RNA delivery systems. Examples of appropriate virus-based gene transfer vectors include, but are not limited to, pCEP4 and pREP4 vectors from Invitrogen, and, more generally, those derived from retrovirises, for example Moloney murine leukemia-virus based vectors such as LX, LNSX, LNCX or LXSN (Miller and Rosman, 1989, Biotechniques 7:980-989); lentiviruses, for example human immunodeficiency virus (“HIV”), feline leukemia virus (“FIV”) or equine infectious anemia virus (“EIAV”)-based vectors (Case et al., 1999, Proc, Natl. Acad. Sci. U.S.A. 96: 22988-2993; Curran et al., 2000, Mol. Ther. 1:31-38; Olsen, 1998, Gene Ther. 5:1481-1487; U.S. Pat. Nos. 6,255,071 and 6,025,192); adenoviruses (Zhang, 1999, Cancer Gene Ther. 6:113-138; Connelly, 1999, Curr. Opin. Mol. Ther. 1:565-572; Stratford-Perricaudet, 1990, Human Gene Ther. 1:241-256; Rosenfeld, 1991, Science 252:431-434; Wang et al., 1991, Adv. Exp. Med. Biol. 309:61-66; Jaffe et al., 1992, Nat. Genet. 1:372-378; Quantin et al., 1992, Proc. Natl. Acad. Sci, U.S.A. 89:2581-2584; Rosenfeld et al., 1992, Cell 68:143-155; Mastrangeli et al., 1993, J. Clin. Invest. 91:225-234; Ragot et al., 1993, Nature 361:647-650; Hayaski et al., 1994, J. Biol. Chem. 269:23872-23875; Bett et al., 1994, Proc. Natl. Acad. Sci. U.S.A. 91:8802-8806), for example Ad5/CMV-based E1-deleted vectors (Li et al., 1993, Human Gene Ther. 4:403-409); adeno-associated viruses, for example pSub201-based AAV2-derived vectors (Walsh et al., 1992, Proc. Natl. Acad. Sci. U.S.A. 89:7257-7261); herpes simplex viruses, for example vectors based on HSV-1 (Geller and Freese, 1990, Proc. Natl. Acad. Sci. U.S.A. 87:1149-1153); baculoviruses, for example AcMNPV-based vectors (Boyce and Bucher, 1996, Proc. Natl. Acad. Sci. U.S.A. 93:2348-2352); SV40, for example SVluc (Strayer and Milano, 1996, Gene Ther. 3:581-587); Epstein-Barr viruses, for example EBV-based replicon vectors (Hambor et al., 1988, Proc. Natl. Acad. Sci. U.S.A. 85:4010-4014); alphaviruses, for example Semliki Forest virus- or Sindbis virus-based vectors (Polo et al., 1999, Proc. Natl. Acad. Sci. U.S.A. 96:4598-4603); vaccinia viruses, for example modified vaccinia virus (MVA)-based vectors (Sutter and Moss, 1992, Proc. Natl. Acad. Sci. U.S.A. 89:10847-10851) or any other class of viruses that can efficiently transduce cells and that can accommodate the CAD11-encoding nucleic acid and sequences necessary and/or desirable for its expression.

Where CAD11 protein levels in a cell are to be increased by direct administration of CAD11 protein to a cell, the CAD11 protein is preferably comprised in a structure that facilitates uptake by a cell, preferably a hair follicle cell, more preferably a dermal papilla cell. For example, CAD11 protein may be comprised in a liposome, microsphere or microbead. CAD11 protein, optionally contained in a liposome, microsphere, or microbead, may be introduced into hair follicles in vivo via topical application or local injection, or ex vivo (see infra).

Administration of CAD11-encoding nucleic acid (optionally contained in a vector molecule) or CAD11 protein may be achieved by methods known in the art, including topical, intracutaneous, intravenous, or oral administration. In preferred non-limiting embodiments of the invention, administration is targeted to hair follicle cells to be treated. In one set of non-limiting embodiments, targeting may be achieved by route and/or site of administration, for example, topical application or injection into an area where promotion of hair growth is desired. In another set of non-limiting embodiments, targeting may be achieved by promoter selection, for example, operably linking a CAD11 nucleic acid to a promoter selectively active in hair follicle cells or an inducible promoter, where the inducing agent is selectively applied to hair follicle cells (e.g., topically or systemically administered CAD11 nucleic acid is operably linked to a tetracycline-inducible promoter, and promoter activity is induced by systemic administration or topical application of tetracycline). CAD11 nucleic acid may also be introduced into hair follicle cells ex vivo (see, infra).

One or more agent that directly increases CAD11 may be administered as a single application or as multiple applications over a period of time. Administration of agent may be, for example and not by way of limitation, once or twice daily, once or twice weekly, once a month, twice a month, once every two months, or once a year. Alternatively, the agent(s) may be administered for a treatment period, followed by a period of no treatment, followed by another treatment period, and this cycle may be repeated. In a specific, non-limiting embodiment of the invention, the interval between treatments or treatment periods may be the average length of time, in the subject to be treated, for a hair follicle to pass through one growth cycle.

The present invention provides for the introduction of CAD11 nucleic acid operably linked to a promoter element and optionally contained in a vector, and/or CAD11 protein, into hair follicles or components thereof maintained in culture (i.e., ex vivo CAD11 gene and/or protein delivery). Hair follicles/dermal papillae may be harvested from a human subject or a non-human animal by methods known in the art or by improvements thereof, as they become available. Non-limiting methods of harvesting hair follicles include harvesting a “plug” of hair bearing tissue removed by an appropriate surgical instrument such as a punch e.g., a standard 4 mm punch. Alternatively, a multiple-strip method of harvesting hair follicles may be accomplished by passing a series of parallel scalpel blades (the multi-bladed knife) through the donor area. A more recent development utilizing single strip harvesting, by removal of a section of tissue either with two parallel blades forming a single long, thin strip or with a single blade producing a long, thin oval, may also be used. Surgery for harvesting hair follicles may be performed with or without the aid of a dissection microscope and encompasses both standard surgical as well as microsurgery or microdissection procedures. Hair follicles/dermal papillae may be maintained in culture, for example as described by Philpott et al., (Dermatol. Clin. 1996 14(4):595-607). CAD11-encoding nucleic acid, as set forth above, may be introduced into the cells of the cultured hair follicles/dermal papillae using methods known in the art, such as transfection of an isolated DNA or RNA by liposomes or other chemically mediated methods, transduction by means of a replicating or non-replicating viral vector, electroporation, biolistic gene delivery, microporation (Bramson et al., 2003, Gene Ther. 10(3):251-260), etc. Gene delivery to a hair follicle may result in transient expression of the delivered gene which may require several applications for effective dosage. Alternatively, the gene may be delivered by a method which causes its permanent incorporation into the genome of the target cell population so as to constitutively express in the target cells and its progeny. Further, CAD11 protein may be introduced into cells of cultured hair follicles/dermal papillae, for example, where the protein is contained in liposomes or microspheres or on microbeads which are internalized by hair follicle cells. In non-limiting embodiments, CAD11 nucleic acid and/or protein may be introduced into dermal papilla cells in culture, whereby the period of inductivity of at least a portion of the cultured dermal papilla cells is prolonged, and/or the inductivity of at least a portion of the dermal papilla cells is enhanced or at least partially restored (where “inductivity” refers to the ability of dermal papilla cells to induce the formation of a hair follicle).

The foregoing methods of directly increasing levels of CAD11 may be used to promote hair growth in a subject in need of such treatment. In non-limiting embodiments, such methods may be used to treat alopecia in a human subject, such as occurs, for example, in male pattern baldness. In other embodiments, such methods may be used to promote hair growth in a domesticated animal which is a laboratory animal, farm animal, pet, or which is used in the wool or fur industries.

The present invention likewise comprises compositions that may be used in the above methods, for example, compositions comprising CAD11 nucleic acid operably linked to a promoter element, optionally contained in a vector, where the composition is a topical formulation or a pharmaceutical preparation suitable for injection; compositions comprising CAD11 protein, optionally contained in a liposome, microsphere, or microbead, where the composition is a topical formulation or a pharmaceutical preparation suitable for injection. Topical formulations include creams, lotions, ointments, etc. Injectable formulations include but are not limited to saline solutions. A composition comprising CAD11 nucleic acid and/or protein may further comprise a permeability-enhancing agent such as dimethylsulfoxide, lipofectamine, oligofectamiine, nanoparticles, and/or cyclofectin. In still further embodiments, the present invention provides for hair follicle cells into which CAD11 nucleic acid and/or protein has been introduced ex vivo.

### Methods of Indirectly Increasing CAD11 in a Subject

The present invention further provides for methods which indirectly increase CAD11 mRNA and/or protein. Such methods do not administer CAD11-encoding nucleic acid or CAD11 protein, but rather administer an agent which results in an increase in CAD11 mRNA transcription, its translation into protein, and/or the half-life and/or functional activity of CAD11 mRNA or CAD11 protein. Agents that may be used to indirectly increase CAD11 in a cell, preferably a hair follicle cell, more preferably a dermal papilla cell, may be identified using the assay systems set forth below. The agent may be, for example but not by way of limitation, a small molecule (e.g., a member of a library developed through combinatorial chemistry), a peptide, a protein, a nucleic acid, a lipid, or a carbohydrate.

Administration of one or more agent that indirectly increases CAD11 (or a direct agent and one or more indirect agents) may be achieved by methods known in the art, including topical, intracutaneous, intravenous, or oral administration. In preferred non-limiting embodiments of the invention, administration is targeted to hair follicle cells to be treated. The one or more agent may be comprised in a composition together with suitable pharmaceutical carriers. The agent may optionally be comprised in a microstructure such as a liposome or microsphere. A composition comprising the agent(s) may further comprise a permeability-enhancing agent such as dimethylsulfoxide, lipofectamine, oligofectamine, nanoparticles, and/or cyclofectin.

One or more agent may be introduced into hair follicle cells ex vivo, for example as set forth in the preceding section.

One or more agent may be administered as a single application or as multiple applications over a period of time. Administration of agent may be, for example and not by way of limitation, once or twice daily, once or twice weekly, once a month, twice a month, once every two months, or once a year. Alternatively, the agent(s) may be administered for a treatment period, followed by a period of no treatment, followed by another treatment period, and this cycle may be repeated. In a specific, non-limiting embodiment of the invention, the interval between treatments or treatment periods may be the average length of time, in the subject to be treated, for a hair follicle to pass through one growth cycle.

The foregoing methods of indirectly increasing levels of CAD11 by administering an effective amount of one or more agent may be used to promote hair growth in a subject in need of such treatment. In non-limiting embodiments, such methods may be used to treat alopecia in a human subject, such as occurs, for example, in male pattern baldness. In other embodiments, such methods may be used to promote hair growth in a domesticated animal which is a laboratory animal, farm animal, pet, or which is used in the wool or fur industries.

### EXAMPLES

### Example 1

### CAD11-EGFP Transgene is Expressed at the at the Borders of Transformed HaCaT Cells

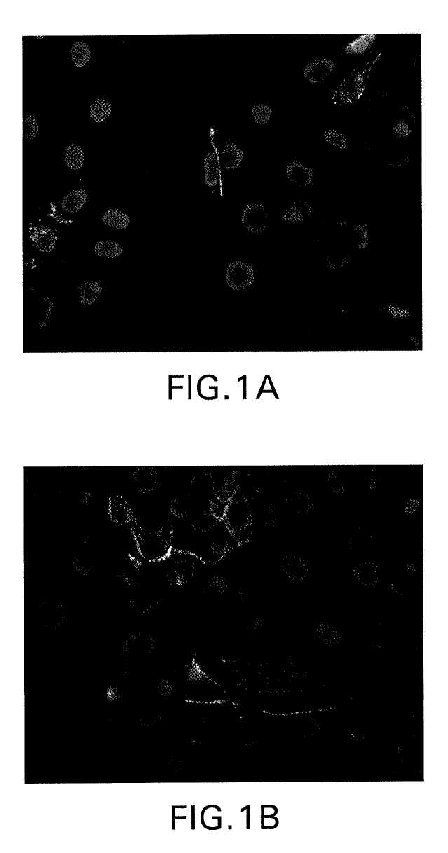

To evaluate the role of CAD11 in hair growth, an expression construct encoding a CAD11-EGFP fusion protein was transfected into HaCaT cells. The human keratinocyte cell line HaCaT is a human keratinocyte cell line which expresses essentially all epidermal differentiation markers but exhibits deficiencies in tissue organization (Maas-Szabowski et al., 2003, J. Cell Sci. 116:2937-2948). Whereas tissue differentiation by normal keratinocytes (NEKs) is regulated by stromal interactions, this mechanism is impaired in HaCaT cells (Id.). As shown in the photomicrographs of FIG. 1A-B, CAD11, as detected by its green fluorescent marker, accumulated at the borders of the HaCaT cells.

### Example 2

### CAD11 is Expressed in Both Rat and Human Dermal Papillae

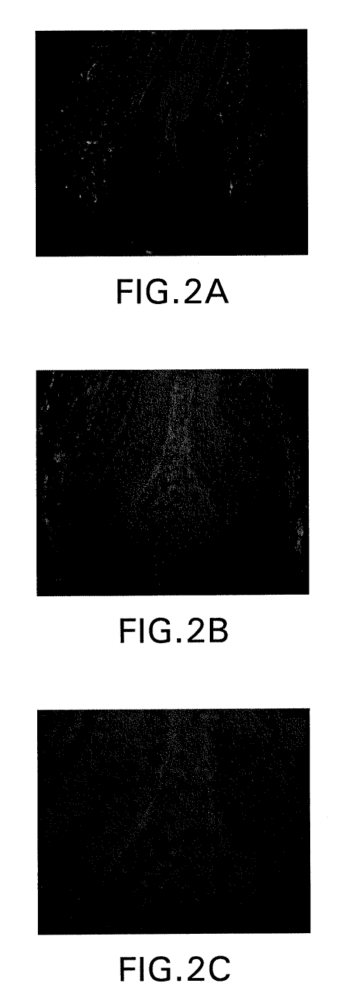

Immunohistochemical staining of sectioned rat vibrissae with labeled CAD11 antibody showed that CAD11 exists as a gradient in the dermal papillae of the rat vibrissae in the anagen phase of the hair growth cycle (FIG. 2A-C).

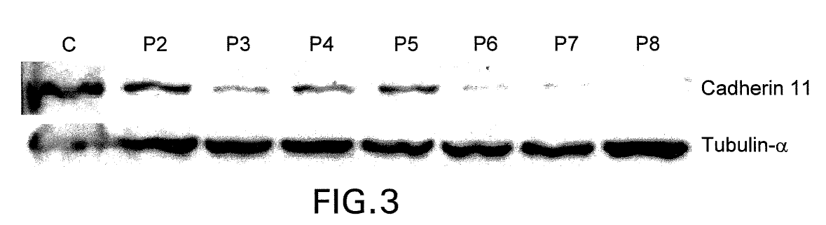

Interestingly, when the level of CAD11 protein was measured in human dermal papillae, serially passaged in culture, it was found that the level of CAD11protein decreased over time (FIG. 3). This decrease correlates with a decrease in the ability of cultured dermal papillae to induce the formation of hair follicles. As the dermal papillae are the “driving force” of hair growth, this data is consistent with a role for CAD11 in hair growth.

### Example 3

### CAD11 is Expressed in Developing Dermal Papillae of Embryonic and Postnatal Rat

Immunohistochemical staining of rat tissue with labeled CAD11 antibody showed that CAD11 exists in the developing dermal papillae of the embryonic rat (FIG. 4A-D). Following birth, the dermal papillae exhibit an increase in CAD11 expression as the derma papillae enter the anagen phase of hair growth (FIG. 5A-F).

Various publications are cited herein, the contents of which are hereby incorporated by reference in their entireties.

1-40. (canceled)

41. A method of inhibiting hair growth comprising administering an agent to a subject, wherein the administered agent elicits a decrease in the level of a cadherin-11 (CAD11) in a cell.

42. The method of claim 41, wherein the CAD11 is a gene comprising a nucleic acid sequence as set forth in Seq. ID. No.: 1, and wherein the agent decreases the level of CAD11 mRNA in a cell.

43. The method of claim 41, wherein the CAD11 is a protein comprising an amino acid sequence as set forth in Seq. ID. No.: 2, and wherein the agent decreases the level of CAD11 protein in a cell.

44. The method of claim 41, wherein the CAD11 is a protein comprising an amino acid sequence as set forth in Seq. ID. No.: 2, and wherein the agent decreases the level of functionally active CAD11 protein in a cell.

45. The method of claim 41, wherein the agent is a CAD11 antisense molecule.

46. The method of claim 41, wherein the agent is a catalytic nucleic acid.

47. The method of claim 41, wherein the agent is a ribozyme.

48. The method of claim 41, wherein the agent is an siRNA.

49. The method of claim 41, wherein the cell is a hair follicle cell.

50. The method of claim 41, wherein the cell is a derma papilla cell.

51. The method of claim 41, wherein the subject is a mammal.

52. The method of claim 41, wherein the subject is a human.

53. The method of claim 41, wherein the subject is a non-human animal selected from the group consisting of a domesticated animal and an animal used in the fur industry.

54. A method of promoting hair growth comprising administering an agent to a subject, wherein the administered agent elicits an increase in the level of a cadherin-11 (CAD11) in a cell.

55. The method of claim 54, wherein the agent is a nucleic acid comprising a nucleic acid having a sequence selected from the group consisting of SEQ ID NO:5, a sequence at least 90 percent homologous to SEQ ID NO:5, and a nucleic acid sequence that encodes a protein having amino acid sequence SEQ ID NO:2.

56. The method of claim 54, wherein the agent is a protein comprising an amino acid sequence as set forth in SEQ. ID. No.: 2, and wherein the agent increases the level of CAD11 protein in a cell.

57. The method of claim 54, wherein the cell is a hair follicle cell.

58. The method of claim 54, wherein the cell is a dermal papilla cell.

59. The method of claim 54, wherein the subject is a human.

60. The method of claim 54, wherein the subject is a non-human animal selected from the group consisting of a domesticated animal, a farm animal, a companion animal, and a laboratory animal.


SEQUENCE LISTING (5 sequences)

SEQ ID NO: 1 - DNA, 3654 bp, Homo sapiens
agatgccgcgggggccgctcgcagccgccgctgacttgtgaatgggaccgggactggggccgggactgacaccgcagcgcttgccctgcgccagggactggcggctcggaggttgcgtccaccctcaagggccccagaaatcactgtgttttcagctcagcggccctgtgacattccttcgtgttgtcatttgttgagtgaccaatcagatgggtggagtgtgttacagaaattggcagcaagtatccaatgggtgaagaagaagctaactggggacgtgggcagccctgacgtgatgagctcaaccagcagagacattccatcccaagagaggtctgcgtgacgcgtccgggaggccaccctcagcaagaccaccgtacagttggtggaaggggtgacagctgcattctcctgtgcctaccacgtaaccaaaaatgaaggagaactactgtttacaagccgccctggtgtgcctgggcatgctgtgccacagccatgcctttgccccagagcggcgggggcacctgcggccctccttccatgggcaccatgagaagggcaaggaggggcaggtgctacagcgctccaagcgtggctgggtctggaaccagttcttcgtgatagaggagtacaccgggcctgaccccgtgcttgtgggcaggcttcattcagatattgactctggtgatgggaacattaaatacattctctcaggggaaggagctggaaccatttttgtgattgatgacaaatcagggaacattcatgccaccaagacgttggatcgagaagagagagcccagtacacgttgatggctcaggcggtggacagggacaccaatcggccactggagccaccgtcggaattcattgtcaaggtccaggacattaatgacaaccctccggagttcctgcacgagacctatcatgccaacgtgcctgagaggtccaatgtgggaacgtcagtaatccaggtgacagcttcagatgcagatgaccccacttatggaaatagcgccaagttagtgtacagtatcctcgaaggacaaccctatttttcggtggaagcacagacaggtatcatcagaacagccctacccaacatggacagggaggccaaggaggagtaccacgtggtgatccaggccaaggacatgggtggacatatgggcggactctcagggacaaccaaagtgacgatcacactgaccgatgtcaatgacaacccaccaaagtttccgcagagcgtataccagatgtctgtgtcagaagcagccgtccctggggaggaagtaggaagagtgaaagctaaagatccagacattggagaaaatggcttagtcacatacaatattgttgatggagatggtatggaatcgtttgaaatcacaacggactatgaaacacaggagggggtgataaagctgaaaaagcctgtagattttgaaaccaaaagagcctatagcttgaaggtagaggcagccaacgtgcacatcgacccgaagtttatcagcaatggccctttcaaggacactgtgaccgtcaagatctcagtagaagatgctgatgagccccctatgttcttggccccaagttacatccacgaagtccaagaaaatgcagctgctggcaccgtggttgggagagtgcatgccaaagaccctgatgctgccaacagcccgataaggtattccatcgatcgtcacactgacctcgacagatttttcactattaatccagaggatggttttattaaaactacaaaacctctggatagagaggaaacagcctggctcaacatcactgtctttgcagcagaaatccacaatcggcatcaggaagccaaagtcccagtggccattagggtccttgatgtcaacgataatgctcccaagtttgctgccccttatgaaggtttcatctgtgagagtgatcagaccaagccactttccaaccagccaattgttacaattagtgcagatgacaaggatgacacggccaatggaccaagatttatcttcagcctaccccctgaaatcattcacaatccaaatttcacagtcagagacaaccgagataacacagcaggcgtgtacgcccggcgtggagggttcagtcggcagaagcaggacttgtaccttctgcccatagtgatcagcgatggcggcatcccgcccatgagtagcaccaacaccctcaccatcaaagtctgcgggtgcgacgtgaacggggcactgctctcctgcaacgcagaggcctacattctgaacgccggcctgagcacaggcgccctgatcgccatcctcgcctgcatcgtcattctcctggtcattgtagtattgtttgtgaccctgagaaggcaaaagaaagaaccactcattgtctttgaggaagaagatgtccgtgagaacatcattacttatgatgatgaagggggtggggaagaagacacagaagcctttgatattgccaccctccagaatcctgatggtatcaatggatttatcccccgcaaagacatcaaacctgagtatcagtacatgcctagacctgggctccggccagcgcccaacagcgtggatgtcgatgacttcatcaacacgagaatacaggaggcagacaatgaccccacggctcctccttatgactccattcaaatctacggttatgaaggcaggggctcagtggccgggtccctgagctccctagagtcggccaccacagattcagacttggactatgattatctacagaactggggacctcgttttaagaaactagcagatttgtatggttccaaagacacttttgatgacgattcttaacaataacgatacaaatttggccttaagaactgtgtctggcgttctcaagaatctagaagatgtgtaaacaggtatttttttaaatcaaggaaaggctcatttaaaacaggcaaagttttacagagaggatacatttaataaaactgcgaggacatcaaagtggtaaatactgtgaaataccttttctcacaaaaaggcaaatattgaagttgtttatcaacttcgctagaaaaaaaaaacacttggcatacaaaatatttaagtgaaggagaagtctaacgctgaactgacaatgaagggaaattgtttatgtgttatgaacatccaagtctttcttcttttttaagttgtcaaagaagcttccacaaaattagaaaggacaacagttctgagctgtaatttcgccttaaactctggacactctatatgtagtgcatttttaaacttgaaatatataatattcagccagcttaaacccatacaatgtatgtacaatacaatgtacaattatgtctcttgagcatcaatcttgttactgctgattcttgtaaatctttttgcttctactttcatcttaaactaatacgtgccagatataactgtcttgtttcagtgagagacgccctatttctatgtcatttttaatgtatctatttgtacaattttaaagttcttattttagtatacgtataaatatcagtattctgacatgtaagaaaatgttacggcatcacacttatattttatgaacattgtactgttgctttaatatgagcttcaatataagaagcaatctttgaaataaaaaaagatttttttttaaaaaaaa

SEQ ID NO: 2 - protein, 796 aa, Homo sapiens
MKENYCLQAALVCLGMLCHSHAFAPERRGHLRPSFHGHHEKGKEGQVLQRSKRGWVWNQFFVIEEYTGPDPVLVGRLHSDIDSGDGNIKYILSGEGAGTIFVIDDKSGNIHATKTLDREERAQYTLMAQAVDRDTNRPLEPPSEFIVKVQDINDNPPEFLHETYHANVPERSNVGTSVIQVTASDADDPTYGNSAKLVYSILEGQPYFSVEAQTGIIRTALPNMDREAKEEYHVVIQAKDMGGHMGGLSGTTKVTITLTDVNDNPPKFPQSVYQMSVSEAAVPGEEVGRVKAKDPDIGENGLVTYNIVDGDGMESFEITTDYETQEGVIKLKKPVDFETKRAYSLKVEAANVHIDPKFISNGPFKDTVTVKISVEDADEPPMFLAPSYIHEVQENAAAGTVVGRVHAKDPDAANSPIRYSIDRHTDLDRFFTINPEDGFIKTTKPLDREETAWLNITVFAAEIHNRHQEAKVPVAIRVLDVNDNAPKFAAPYEGFICESDQTKPLSNQPIVTISADDKDDTANGPRFIFSLPPEIIHNPNFTVRDNRDNTAGVYARRGGFSRQKQDLYLLPIVISDGGIPPMSSTNTLTIKVCGCDVNGALLSCNAEAYILNAGLSTGALIAILACIVILLVIVVLFVTLRRQKKEPLIVFEEEDVRENIITYDDEGGGEEDTEAFDIATLQNPDGINGFIPRKDIKPEYQYMPRPGLRPAPNSVDVDDFINTRIQEADNDPTAPPYDSIQIYGYEGRGSVAGSLSSLESATTDSDLDYDYLQNWGPRFKKLADLYGSKDTFDDDS

SEQ ID NO: 3 - protein, 9 aa, Artificial Sequence
  Description of Artificial Sequence Synthetic Peptide
DXNDNAPXF

SEQ ID NO: 4 - protein, 5 aa, Artificial Sequence
  Description of Artificial Sequence Synthetic Peptide
DXNDN

SEQ ID NO: 5 - DNA, 2388 bp, Homo sapiens
atgaaggagaactactgtttacaagccgccctggtgtgcctgggcatgctgtgccacagccatgcctttgccccagagcggcgggggcacctgcggccctccttccatgggcaccatgagaagggcaaggaggggcaggtgctacagcgctccaagcgtggctgggtctggaaccagttcttcgtgatagaggagtacaccgggcctgaccccgtgcttgtgggcaggcttcattcagatattgactctggtgatgggaacattaaatacattctctcaggggaaggagctggaaccatttttgtgattgatgacaaatcagggaacattcatgccaccaagacgttggatcgagaagagagagcccagtacacgttgatggctcaggcggtggacagggacaccaatcggccactggagccaccgtcggaattcattgtcaaggtccaggacattaatgacaaccctccggagttcctgcacgagacctatcatgccaacgtgcctgagaggtccaatgtgggaacgtcagtaatccaggtgacagcttcagatgcagatgaccccacttatggaaatagcgccaagttagtgtacagtatcctcgaaggacaaccctatttttcggtggaagcacagacaggtatcatcagaacagccctacccaacatggacagggaggccaaggaggagtaccacgtggtgatccaggccaaggacatgggtggacatatgggcggactctcagggacaaccaaagtgacgatcacactgaccgatgtcaatgacaacccaccaaagtttccgcagagcgtataccagatgtctgtgtcagaagcagccgtccctggggaggaagtaggaagagtgaaagctaaagatccagacattggagaaaatggcttagtcacatacaatattgttgatggagatggtatggaatcgtttgaaatcacaacggactatgaaacacaggagggggtgataaagctgaaaaagcctgtagattttgaaaccaaaagagcctatagcttgaaggtagaggcagccaacgtgcacatcgacccgaagtttatcagcaatggccctttcaaggacactgtgaccgtcaagatctcagtagaagatgctgatgagccccctatgttcttggccccaagttacatccacgaagtccaagaaaatgcagctgctggcaccgtggttgggagagtgcatgccaaagaccctgatgctgccaacagcccgataaggtattccatcgatcgtcacactgacctcgacagatttttcactattaatccagaggatggttttattaaaactacaaaacctctggatagagaggaaacagcctggctcaacatcactgtctttgcagcagaaatccacaatcggcatcaggaagccaaagtcccagtggccattagggtccttgatgtcaacgataatgctcccaagtttgctgccccttatgaaggtttcatctgtgagagtgatcagaccaagccactttccaaccagccaattgttacaattagtgcagatgacaaggatgacacggccaatggaccaagatttatcttcagcctaccccctgaaatcattcacaatccaaatttcacagtcagagacaaccgagataacacagcaggcgtgtacgcccggcgtggagggttcagtcggcagaagcaggacttgtaccttctgcccatagtgatcagcgatggcggcatcccgcccatgagtagcaccaacaccctcaccatcaaagtctgcgggtgcgacgtgaacggggcactgctctcctgcaacgcagaggcctacattctgaacgccggcctgagcacaggcgccctgatcgccatcctcgcctgcatcgtcattctcctggtcattgtagtattgtttgtgaccctgagaaggcaaaagaaagaaccactcattgtctttgaggaagaagatgtccgtgagaacatcattacttatgatgatgaagggggtggggaagaagacacagaagcctttgatattgccaccctccagaatcctgatggtatcaatggatttatcccccgcaaagacatcaaacctgagtatcagtacatgcctagacctgggctccggccagcgcccaacagcgtggatgtcgatgacttcatcaacacgagaatacaggaggcagacaatgaccccacggctcctccttatgactccattcaaatctacggttatgaaggcaggggctcagtggccgggtccctgagctccctagagtcggccaccacagattcagacttggactatgattatctacagaactggggacctcgttttaagaaactagcagatttgtatggttccaaagacacttttgatgacgattct